(19) United States
(12) Patent Application Publication
(10) Pub. No.: US 2025/0213231 A1
(43) Pub. Date: Jul. 3, 2025

(54) BOWEL TRACKER SYSTEM FOR PASSIVE MONITORING OF BOWEL HABITS

(71) Applicants: CEDARS-SINAI MEDICAL CENTER, Los Angeles, CA (US); The Regents of The University of California, Oakland, CA (US)

(72) Inventors: David Ziring, Sherman Oaks, CA (US); Edward Jay Wang, La Jolla, CA (US); Yinan Xuan, La Jolla, CA (US)

(73) Assignees: CEDARS-SINAI MEDICAL CENTER, Los Angeles, CA (US); The Regents of The University of California, Oakland, CA (US)

(21) Appl. No.: 18/835,075

(22) PCT Filed: Feb. 2, 2023
(86) PCT No.: PCT/US2023/061863

§ 371 Date: Aug. 1, 2024

Related U.S. Application Data
(60) Provisional application No. 63/305,803, filed on Feb. 2, 2022.

(51) Int. Cl. A61B 10/00; A61B 5/00; A61B 5/11

(52) U.S. Cl. CPC A61B 10/0038; A61B 5/0022; A61B 5/1116; A61B 5/7267; A61B 2562/0219

(57) ABSTRACT

Bowel tracker systems including a wearable device and an app installed on a mobile device wirelessly connected to the wearable device, and methods of monitoring bowel habits of a subject by a bowel tracker system are provided. A wearable device to be worn by a user includes an accelerometer configured to detect changes in positions of the user wearing the wearable device; a light sensor configured to detect changes in intensities of light; and a transmitter configured to communicate with an external device. The wearable device detects when the user is sitting with the user's lower body undressed based on the changes detected by the accelerometer and light sensor; captures stool frequency data based on the detected sitting by the user; and transmits the captured stool frequency data to the external device. A mobile app installed on the external device records the log of the stool frequency data.

### CROSS-REFERENCE TO RELATED APPLICATIONS

This application claims the benefit under 35 U.S.C. § 119(e) of U.S. provisional patent application No. 63/305,803, filed Feb. 2, 2022, the entirety of which is hereby incorporated by reference.

### FIELD OF INVENTION

The present invention relates to a bowel tracker system including a wearable device including an accelerometer, a light sensor, and a transmitter, and more specifically, to a thigh-worn tag for passive monitoring of bowel habits of a subject.

### BACKGROUND

Stool frequency is a key patient reported outcome (PRO) and its measurement is essential for the evaluation of promising therapeutic agents in the care of patients with Irritable bowel syndrome (IBS), Crohn's disease and ulcerative colitis. For example, IBS causes patients to defecate very often. It is important for patients and physicians to track the frequency, duration, and timing of the patients' restroom usage, as well as the shape of the feces. It indicates disease activity, and is a key component of several prognostic scoring indices (e.g. in those patients hospitalized with acute severe colitis). However, memorization of that information places a heavy cognitive load on the patients. Unfortunately, the capture of stool frequency data is plagued by recall bias. Noting stool frequency in a written diary is inaccurate and impractical. It is often not obtained contemporaneously, and anecdotally patients have been witnessed to complete a one-week stool frequency diary prior to their appointments in the waiting room of the clinical trials research center. Even inpatient nurses often are not able to accurately capture stool frequency data on their hospitalized patients. Therefore, there is a large unmet need to capture this data accurately and with precision in order to more objectively evaluate disease activity and in turn to better evaluate the efficacy of new therapeutics.

Described herein is a bowel tracker system including a wearable device and a method of capturing stool frequency data passively using wearable technology to solve the issues identified above. This wearable solution is configured to capture data for a specific period of time, typically seven days, that comprises clinical activity indices such as Crohn's disease activity index (CDAI) and Mayo score. Further, this solution would have application across a range of gastrointestinal (GI) diseases, for instance, in irritable bowel syndrome (IBS) with constipation or diarrhea.

### SUMMARY OF THE INVENTION

The present disclosure provides a wearable device to be worn by a user. According to various embodiments of the present invention, the wearable device includes an accelerometer configured to detect changes in positions of the user wearing the wearable device; a light sensor configured to detect changes in intensities of light; and a transmitter configured to communicate with an external device. The wearable device is configured to detect when the user is sitting with the user's lower body undressed based on the changes detected by the accelerometer and light sensor; capture stool frequency data based on the detected sitting by the user; and transmit the captured stool frequency data to the external device.

In some embodiments, the wearable device further includes a processor configured to receive data including the detected changes from the accelerometer and light sensor; and determine whether the user is sitting on a toilet based on the received data.

In some embodiments, the processor is further configured to capture stool frequency, timing, and duration of the user.

For example, the wearable device includes a tag worn on a thigh of the user. For example, the tag is worn at an upper portion of the thigh.

In some embodiments, the tag is less than 0.5 cm thick. For example, the transmitter includes a Bluetooth transmitter configured to communicate with the external device paired with the Bluetooth transmitter and/or positioned within a specified distance from the wearable device.

In some embodiments, the user is a patient with a bowel disorder or gastrointestinal disease. For example, the bowel disorder includes Crohn's disease, ulcerative colitis, and functional bowel disorders (FBD) including irritable bowel syndrome and functional dyspepsia.

In some embodiments, the wearable device further includes a microphone. The processor is further configured to receive acoustic data sensed by the microphone and/or acoustic data sensed by a microphone of the external device; and determine whether the user is sitting on the toilet based on the received acoustic data in addition to the changes detected by the accelerometer and light sensor.

In some embodiments, the acoustic data are robustly identified using a neural network model tuned for acoustic classification running on the external device. In some embodiments, a neural network model utilizes transfer learning to first pre-train a convolutional neural network (CNN) with a large corpus of labeled acoustics data.

In some embodiments, the wearable device further includes an audio output unit or a speaker configured to output a notification when the processor determines that the user has been in a seating position for more than a preset period of time.

In some embodiments, the wearable device further includes an input unit or a button configured to receive an input from the user in response to the notification output from the audio output unit or speaker, the input confirming that the user is actually having a bowel movement.

In some embodiments, the processor is further configured to train algorithm for demarcating bowel movement events in response to the input, using machine learning and statistical modeling, verification performed by the external device.

In some embodiments, the processor is further configured to cause the transmitter to transmit the captured stool frequency, timing, and duration of the user to the external device comprising a smartwatch or smartphone.

In some embodiments, the external device is configured to execute an application to process the captured stool frequency data received from the wearable device.

In some embodiments, the external device includes a display and is further configured to initiate a push notification to the user, noting a bowel movement was detected; and present an ecological momentary assessment (EMA) on the display.

The present disclosure also provides a method for monitoring bowel habits of a subject by a wearable device worn by the subject. According to various embodiments of the present invention, the method includes detecting, by an accelerometer of the wearable device, changes in positions of the subject; detecting, by a light sensor of the wearable device, changes in intensities of light; detecting, by a processor of the wearable device, when the subject is sitting with the subject's lower body undressed based on the changes detected by the accelerometer and light sensor; capturing, by the processor, stool frequency data based on the detected sitting by the subject; and transmitting, via a transmitter of the wearable device, the captured stool frequency data to the external device.

In some embodiments, the method further includes receiving, by the processor, data including the detected changes from the accelerometer and light sensor; and determining, by the processor, whether the subject is sitting on a toilet based on the received data.

In some embodiments, the method further includes capturing stool frequency, timing, and duration of the subject.

For example, the wearable device includes a tag worn on a thigh of the subject. For example, the tag is worn at an upper portion of the thigh.

In some embodiments, the method further includes determining a toilet seated position in response to the accelerometer detecting that the wearable device or tag worn by the subject is substantially parallel to the ground. In some embodiments, a threshold detected by the accelerometer to be determined as the toilet seating position is +/− about 10 degrees parallel to the ground.

In some embodiments, the tag is less than 0.5 cm thick.

For example, the transmitter includes a Bluetooth transmitter configured to communicate with the external device paired with the Bluetooth transmitter and/or positioned within a specified distance from the wearable device.

In some embodiments, the subject is a patient with a bowel disorder or gastrointestinal disease. For example, the bowel disorder includes Crohn's disease, ulcerative colitis, and functional bowel disorders (FBD) comprising irritable bowel syndrome and functional dyspepsia.

In some embodiments, the wearable device further includes a microphone, and the method further includes receiving acoustic data sensed by the microphone and/or acoustic data sensed by a microphone of the external device; and determining whether the subject is sitting on the toilet based on the received acoustic data in addition to the changes detected by the accelerometer and light sensor.

In some embodiments, the acoustic data are robustly identified using a neural network model tuned for acoustic classification running on the external device. In some embodiments, a neural network model utilizes transfer learning to first pre-train a convolutional neural network (CNN) with a large corpus of labeled acoustics data.

In some embodiments, the wearable device further includes an audio output unit or a speaker, and the method further includes outputting, via the audio output unit or speaker, a notification when the processor determines that the subject has been in a seating position for more than a preset period of time.

In some embodiments, the wearable device further includes an input unit or a button, and the method further includes receiving an input from the subject, via the input unit or button, in response to the notification output from the audio output unit or speaker, the input confirming that the subject is actually having a bowel movement.

In some embodiments, the method further includes training algorithm for demarcating bowel movement events in response to the input, using machine learning and statistical modeling, verification performed by the external device.

In some embodiments, the method further includes transmitting, via the transmitter, the captured stool frequency, timing, and duration of the subject to the external device comprising a smartwatch or smartphone.

In some embodiments, the external device is configured to execute an application to process the captured stool frequency data received from the wearable device.

In some embodiments, the external device includes a display and the external device is further configured to initiate a push notification to the subject, noting a bowel movement was detected; and present an ecological momentary assessment (EMA) on the display.

The present disclosure further provides a bowel tracker system for monitoring bowel habits of a subject. In various embodiments of the present invention, the system includes a wearable device to be worn by the subject; and a smart device configured to communicate with the wearable device wirelessly. The wearable device includes an accelerometer configured to detect changes in positions of the subject wearing the wearable device; a light sensor configured to detect changes in intensities of light; and a transmitter configured to communicate with the smart device. The wearable device is configured to detect when the subject is sitting with the subject's lower body undressed based on the changes detected by the accelerometer and light sensor; capture stool frequency data based on the detected sitting by the subject; and transmit the captured stool frequency data to the smart device. The smart device is further configured to execute an application to process the stool frequency data received from the wearable device.

For example, the wearable device includes a tag worn on a thigh of the subject. For example, the smart device includes a smartwatch or smartphone paired with the transmitter and/or positioned within a specified distance from the wearable device.

In some embodiments, the system further includes a toilet tag attachable to a toilet. The toilet tag is configured to communicate with at least one of the wearable device and the smart device. In some embodiments, a distance between the toilet tag attached to the toilet and the wearable device is used to determine a toilet seating position of the subject.

In some embodiments, the subject is a patient with a bowel disorder or gastrointestinal disease. For example, the bowel disorder includes Crohn's disease and ulcerative colitis.

In some embodiments, the wearable device further includes a microphone, and the wearable device is further configured to receive acoustic data sensed by the microphone and/or acoustic data sensed by a microphone of the smart device; and determine whether the subject is sitting on the toilet based on the received acoustic data in addition to the changes detected by the accelerometer and light sensor.

In some embodiments, the acoustic data are robustly identified using a neural network model tuned for acoustic classification running on the smart device. In some embodiments, a neural network model utilizes transfer learning to first pre-train a convolutional neural network (CNN) with a large corpus of labeled acoustics data.

In some embodiments, the wearable device further includes an audio output unit or a speaker configured to output a notification when the subject has been determined to be in a seating position for more than a preset period of time.

In some embodiments, the wearable device further includes an input unit or a button configured to receive an input from the subject in response to the notification output from the audio output unit or speaker, the input confirming that the subject is actually having a bowel movement.

In some embodiments, at least one of the wearable device or the smart device is further configured to train algorithm for demarcating bowel movement events in response to the input, using machine learning and statistical modeling, verification performed by the smart device.

In some embodiments, the transmitter is further configured to transmit the captured stool frequency, timing, and duration of the subject to the smart device.

In some embodiments, the smart device includes a display and the smart device is further configured to initiate a push notification to the subject, noting a bowel movement was detected; and present an ecological momentary assessment (EMA) on the display.

In some embodiments, the smart device is further configured to display visual information on the display in response to the processed stool frequency data, the visual information.

In some embodiments, the visual information includes a visual picker for Bristol Stool Scale and select questionnaire around food/fluid intake and discomfort/pain.

In some embodiments, the captured stool frequency data is transmitted to the smart device to be processed by the smart device and/or to be displayed at the smart device.

In some embodiments, the stool frequency data captured and transmitted by the wearable device and processed by the smart device is presented to the subject by being displayed at the smart device.

In some embodiments, the capturing, transmitting, processing, and displaying the stool frequency data are performed passively or automatically without requiring the subject's direct or manual input via the wearable device and/or smart device.

In some embodiments, the captured stool frequency data is transmitted from the wearable device to the smart device when the wearable device and the smart device are within a threshold distance allowing wireless communication between the wearable device and the smart device.

In some embodiments, when the wearable device and the smart device are not within the threshold distance at the time of capturing the stool frequency data, the wearable device is further configured to transmit the captured stool frequency data to the smart device next time when the wearable device and the smart device are within the threshold distance.

In some embodiments, the wearable device further includes a memory configured to store the captured stool frequency data.

In some embodiments, the captured stool frequency data is stored at the memory until the captured stool frequency data is transmitted to the external device.

In some embodiments, the changes detected by the accelerometer and light sensor includes a change in a light intensity detected by the light sensors exceeds a first threshold and a decrease in a height position of the user detected by the accelerometer exceeds a second threshold.

In some embodiments, the change in the light intensity exceeds the first threshold by a period of at least two seconds or at least three seconds during which period the decrease in the height position also exceeds the second threshold.

In some embodiments, the decrease in the height position corresponds to at least 12 inches or at least 18 inches or at least 20 inches.

In some embodiments, a toilet seated position is detected by the accelerometer when the wearable device or tag worn by the user is substantially parallel to the ground. In some embodiments, a threshold detected by the accelerometer to be determined as the toilet seating position is +/− about 10 degrees parallel to the ground.

In some embodiments, responsive to the acoustic data being indicative of the user having a bowel movement, determining that the user is having a bowel movement regardless of whether the changes detected by at least the light sensor indicate that the user is sitting on the toilet.

In some embodiments, the first threshold is at least 20% or at least 30% or at least 40% or at least 50% or at least 60% or least 70% or at least 80% or at least 90% relative to a light intensity before the detected change.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A shows a bowel tracker system including wearable device and an external device in accordance with various embodiments of the present invention.

FIG. 1B shows components of a wearable device in accordance with various embodiments of the present invention.

FIG. 1C shows a wearable device attached to a thigh of a subject in accordance with various embodiments of the present invention.

FIG. 2 shows a graph showing signal amplitude generated based on data received from an accelerometer and a light sensor of a wearable device in accordance with various embodiments of the present invention.

FIG. 3 shows an audioset dataset including toilet flushing samples in accordance with various embodiments of the present invention.

FIG. 4 shows a screen shot of a notification displayed via a mobile app in accordance with various embodiments of the present invention.

FIG. 5 shows a screen shot of an activity logs page displayed via a mobile app in accordance with various embodiments of the present invention.

FIG. 6 shows an exemplary wearable device/sensor tag attached to a thigh of a subject in accordance with various embodiments of the present invention.

FIG. 7 shows a screen shot of a connection status or connection set up page displayed via a mobile app in accordance with various embodiments of the present invention.

FIG. 8 shows a screen shot of a connection status displayed via a mobile app in accordance with various embodiments of the present invention.

FIG. 9 shows a diagram describing determining a toilet activity or a toilet seating position of a subject in accordance with various embodiments of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

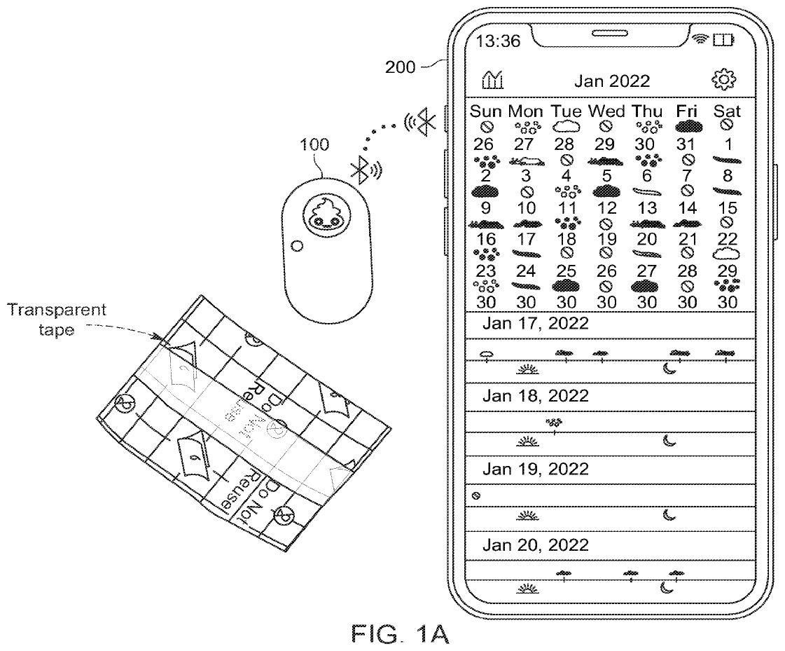

The various embodiments are directed to a bowel tracker system including a wearable device 100 and a mobile app installed on and executed by an external device 200, as shown in FIG. 1A. For example, the external device 200 is wirelessly connected to the wearable device 100. In some embodiments, as exemplified in FIGS. 1B and 1C, the wearable device 100 is a thigh-worn wearable device containing (a) an input button, (b) a light sensor, (c) an accelerometer, (d) a buzzer, (e) a battery, and a transmitter with the augmentation of data quality verified by acoustic processing. In some embodiments, the wearable device 100 does not include all of the above-identified components and may contain various combination of these components. In some embodiments, the wearable device 100 may further contain other types of sensors such as a temperature sensor, a pressure sensor, and/or a level sensor. The temperature sensor measures the amount of heat energy in a source, detecting temperature changes. The pressure sensor reflects the sense changes in gases and liquids. The level sensor is used for detecting the actual level of the substances, the substances including powders, liquid, and granular material.

The wearable device 100 also has a processor or controller operably coupled with other components of the wearable device such as the accelerometer, light sensor, and transmitter to control the other components and receive data from the other components. The wearable device 100 is powered by a battery. For example, the transmitter is a Bluetooth transmitter configured to communicate with a device, such as a mobile device 200, present within a certain distance from the wearable device 100 and/or paired with the Bluetooth transmitter. That is, in some embodiments, the wearable device 100 is a Bluetooth Low-Energy (BLE) sensor tag. In some embodiments, the wearable device is an ultra-thin (<0.5 cm) tag 100, allowing it to be worn discretely under clothing without protruding and snagging. As shown in FIGS. 1A and 1C, in some embodiments, an adhesive/transparent tape is provided and applied over the tag 100 and the skin of the thigh to attach the tag on the thigh.

In various embodiments, a bowel tracker system includes a wearable device 100 or sensor tag and a mobile application (app) installed on an external device 200 such as a smartphone 200a. The mobile app receives real-time data returned from the sensor tag 100 to detect bathroom activities and help the user to log their bathroom activity.

A bowel tracker system in accordance with the various embodiments passively captures stool frequency, timing, and duration of patients with bowel disorders without inherent recall bias by (1) automatically demarcating stool frequency, timing, and duration while (2) promoting just-in-time reflections around coincident symptoms such as bloating, abdominal pain, and stool consistency and the presence of blood. In some embodiments, the wearable device or tag 100 is waterproof, does not need charging, and skin-friendly for continuous wear for the entire observation duration (for example 1 week or 2-4 weeks) when worn on the upper thigh of a subject.

### Light Sensor and Accelerometer

To demarcate bowel movement events accurately and conveniently, the tag 100 is equipped with a light sensor and an accelerometer, which together detect when a person is sitting with their lower body undressed. For example, undressing is detected by the light sensor and the seated position of the person is detected by the accelerometer when the tag 100 worn by the user is substantially parallel to the ground. That is, when the tag 100 is not substantially parallel to the ground, the seated position of the user is not detected. When the person's position is changed from a standing position to the seated position, the orientation of the tag 100 is changed, for example, from a vertical orientation to a horizontal orientation or vice versa. Therefore, the angle between a center line or reference point of the tag 100 and the ground is changed when the person sits on a toilet. In some embodiments, the threshold angle detected by the accelerometer to be determined as toilet seating is +/− about 10 degrees parallel to the ground. In some embodiments, the threshold detected by the accelerometer to be determined as toilet seating is +/−15, 14, 13, 12, 11, 10, 9, 8, 7, 6, or 5 degrees parallel to the ground. This is determined by comparing to a calibration made once the tag 100 is mounted onto the user. During calibration, the user is seated with their leg parallel to the ground.

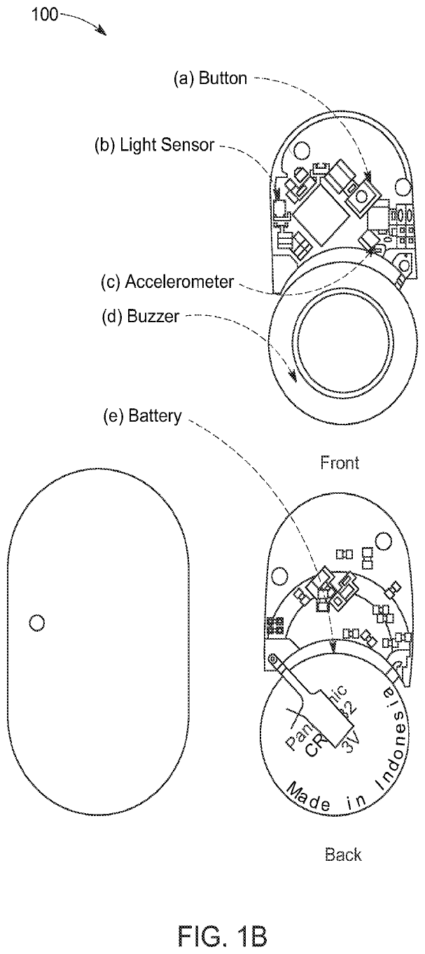

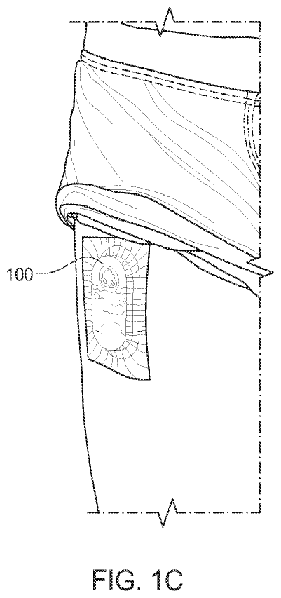

See the exemplary graph of signal amplitude in FIG. 2 showing detection of toilet seating based on the data received from the accelerometer and light sensor. To avoid incorrectly labeling such occurrences, which may be confounded by events such as sitting to urinate or to dress, the tag 100 will interact with the wearer in a low-burden mechanism. To train the algorithm, the wearer will interact with an input device/button of the tag 100. For example, the wearer presses a button on the tag 100, shown in FIG. 1B, to note when they are actually having a bowel movement. In some embodiments, the button on the tag 100 can be used to indicate both positive and negative answers by different pressing patterns. For example, pressing the button once is yes and pressing the button twice is no. This low-burden mechanism is triggered by producing a short and quiet notification jingle with the buzzer speaker on the tag 100, shown in FIG. 1B, after detecting such a sitting event lasting more than a preset period of time, for example, 10 seconds. The preset period of time may be less than 10 seconds, for example, 9, 8, 7, 6, or 5 seconds or more than 10 seconds, for example, 11-15 seconds, 16-20 seconds, or more than 20 seconds. Because the tag 100 would inherently be in an accessible location facing the wearer, the wearer can simply reach down and press the button to confirm the bowel movement after being reminded. In some embodiments, instead of pressing the button of the tag 100, the confirmation is input via the app installed on the external device 200. Once the algorithm is refined using machine learning and statistical modeling, the verification will be performed on an external device 200 such as a smartphone 200a or smartwatch 200b.

The light sensor of the tag 100 detects light when the subject undresses pants or skirts. Light is detected when the difference of the light exceeds a certain threshold value. The threshold value will be different when the subject wears pants and skirts. In some embodiments, the default setting is for detecting light when undressing pants. In some embodiments, the subject changes the default setting via the app, indicating that the subject is wearing skirts. This change in the setting will more accurately calculate the threshold value used for determining toilet seating in consideration of the differences in light when the subject is wearing pants and when the subject is wearing skirts.

Moreover, if the subject undresses in the dark, it would be difficult for the light sensor to detect light. For example, the subject may seat on the toilet at night without turning on the light in the bathroom. In this case, no detection value may be sensed by the light sensor and the absence of the light detection should not be considered to determine toilet seating. In some embodiments, to avoid such false results at night or in the darkness, a different weight is given to the value detected by the light sensor. For example, the weight of the value detected by the light sensor is reduced based on the time of the day such that no weight or less weight is given after sunset. In some embodiments, the subject can set a preferred mode for nighttime via the app such that the value detected by the light sensor is ignored according to the set mode if the subject tends to not turn on the light in the bathroom when seating on the toilet at night. In this case, only the value obtained by the accelerometer is used for determining toilet seating.

The tag 100 logs the duration and time of the bowel movement event. The tag 100 communicates this information to the external device 200 such as the patient's personal smartphone 200a. In one embodiment, the tag 100 communicates with the external device 200 next time when the tag is in proximity to the phone. For example, the tag 100 communicates with the external device 200 wirelessly, using the ultra-low power Bluetooth protocol BLE. When that happens, the smartphone app executed by the smartphone 200a, as exemplified in FIGS. 1A, 4, 5, 7, and 8, will initiate a push notification to the user, noting a bowel movement was detected and presents an ecological momentary assessment (EMA). Because of how frequent users interact with their smartphones 200a, the time between bowel movement and EMA would likely be a just-in-time (JIT) interaction that will lead to high compliance with much better accuracy in recall compared to an end-of-day reflection that is standard for symptom diaries today. In some embodiments, the confirmed bowel movement data may be transmitted to a data server via the smartphone 200a communicating with the data server.

### Acoustic Sensing

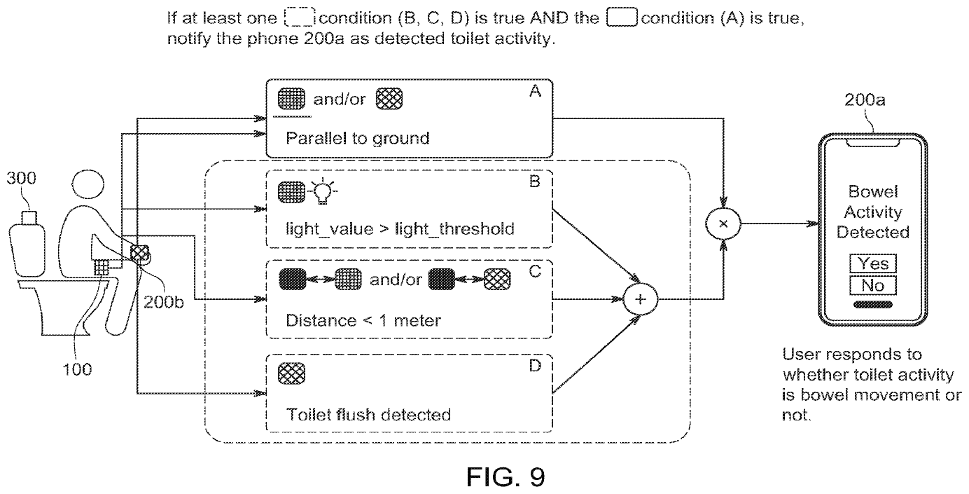

An additional modality that can augment the detection of toilet use is acoustic sensing using a wearable microphone either integrated into the wearable tag 100 directly or leveraging microphones in smartwatches 200b or smartphone 200a. When a seated toilet activity is detected by the tag 100, the microphone will capture the sounds during that period to observe for toilet flushing sound as an additional verification of toilet using behavior. In our feasibility data recorded using a smartwatch 200b, such as Apple Watch, microphone as an external device 200, as exemplified in D of FIG. 9, the toilet flushing sound is clearly captured when the user flushes the toilet. In this case, toilet flushing can be detected even if the user seating on the toilet does not have a smartphone 200a. Further, in case the user is not wearing a smartwatch 200b, the toilet flush can be detected by a microphone integrated into the wearable tag 100 according to some embodiments.

This sound can be robustly identified using a neural network model tuned for acoustic classification running on the mobile phone 200. Such a neural network model would utilize transfer learning to first pre-train a convolutional neural network (CNN) with a large corpus of labeled acoustics data, such as Google Research's opensource Audioset dataset shown in FIG. 2, which is a large collection of 10 second, human-labeled sound data clips from YOUTUBE™. In this dataset, there is over 6 hours of toilet flushing samples, including more than 2000 unique examples. Thus, the acoustic data collected by the microphone together with the data collected by the accelerometer and light sensor can be used to more accurately detect toilet use by the subject wearing the tag 100.

In some embodiments, in case the light sensor cannot detect undressing precisely in the darkness as discussed above, more weight is given to the value detected by the microphone. In some embodiments, toilet seating is determined based on the values detected by the accelerometer and the microphone without considering the value detected by the light sensor during the nighttime or according to the setting input by the subject via the app

### Toilet Proximity Tag

An additional modality that can augment the detection of toilet use in dark settings is the use of an additional BLE tag (toilet tag) 300 placed on the toilet. In some embodiments, the toilet tag 300 is used to capture at-home settings where a toilet may be used in the night. The toilet tag 300 placed on the toilet will be discoverable by the wearable tag 100. Referring to FIG. 9, the wearable tag 100 can measure its proximity to the toilet tag 300 based on the received signal strength (RSSI). The RSSI can be converted to physical distance (meters) using a standard equation of: Distance=10 \hat{ } ((Measured Power−RSSI)/(10*N) where Measured Power is the measured signal strength at 1 meter, RSSI is the current measured signal strength, and N is the environmental factor. N assumes the value of 2 assuming free space.

In some embodiments, in case the light sensor cannot detect undressing precisely in the darkness as discussed above, the distance to the toilet tag 300 is given more weight. In some embodiments, toilet seating is determined based on the values detected by the accelerometer and the proximity to the toilet tag 300 without considering the value detected by the light sensor during the nighttime or according to the setting input by the subject via the app.

### Wearable Sensor for Bowel Passing Activity Tagging

The initial feasibility was completed with the use of a custom sensor tag 100 built with the data collected being directly recorded by a computer and the detection algorithm implemented using a computing programming language such as the Python scientific computing programming language. For example, the code written in Python is converted into the C-programming language to enable on-device posture detection. For example, for the code to work with the commercial sensor tag, such as the one designed by MEEBLUE™, the Firmware API provided by MEEBLUE™ is used to integrate the algorithm into the sensor tag.

### Smartphone Application for Just-in-Time Interaction Ecological Momentary Assessment (EMA) of Bowel Activity

Smartphone-based EMA is well documented in literature as an effective way to capture context of an activity, with the accuracy of the log enhanced by the timely presentation of the assessment. Building on top of the Bluetooth connection app demo from MEEBLUE™, a front facing user interface was developed, drawing inspiration from irritable bowel syndrome (IBS) tracking apps currently available on the App store, including a visual picker for Bristol Stool Scale and select questionnaire around food/fluid intake and discomfort/pain. For example, iOS or Android apps are developed for specific phones to ensure user interface consistency on different phones.

### Smartwatch

Nowadays, smartwatches are popular, and many people wear them. According to various embodiments of the present invention, the inventive bowel tracker system also includes a smartwatch 102b wearable by a user. In some embodiments, the smartwatch 102b is wirelessly connected to a smartphone 102a. Referring to A of FIG. 9, in some embodiments, the toilet seated position of the user is detected by the smartwatch 102b worn by the user. In some embodiments, it is determined that the user is seated at the toilet when the smartwatch 102b is parallel to the ground. In some embodiments, the orientation and position of the smartwatch 102b are calibrated by having the user do standing/sitting action while having the smartwatch 102b on. Preferably, the standing/sitting action is performed by the user at the primary toilet that the user usually uses. Further, referring to D of FIG. 9, the smartwatch 102b picks up sound of toilet flush via a microphone integrated to the smartwatch. Thus, in some embodiments, detection of the toilet seating position and toilet flush by the smartwatch 102b is sufficient to detect the toilet activity of the user wearing the smartwatch 102b. In some embodiments, even if the user is not wearing the sensor tag 100, the smartwatch 102b can detect the toilet activity of the user. Although C of FIG. 9 shows that the distance between the sensor tag 100 and toilet tag 300 is used in determining the toilet activity of the user, in some embodiments, a distance between the smartwatch 102b and the toilet tag 300 can also be used to determine the toilet activity if the user is not wearing the sensor tag 100. Further, in some embodiments, the toilet flush detected by the smartwatch 102b is transmitted to the smartphone 102a.

### Efficacy Study

A real-world patient deployment (N=20) is conducted in two groups of volunteers. The first group wears the sensor tag with a smartphone app that is triggered by the tag 100. The second group does not wear a sensor tag and instead only use the same smartphone app that requires manual logging of toilet activity (marking the estimated start time, duration, and EMA). For 2 weeks, subjects record their activities. Additionally, each evening, the patients are asked to fill out a standard paper diary recalling their bowel frequencies. The smartphone app will not show toilet activities logged. An exit interview around convenience, social acceptability, confidence of log quality, and overall user experience are assessed using both a user survey as well as unstructured interviewing.

Paper form reported bowel activity frequency is expected to be significantly different from app-based recording. Further, wearable assisted logging is expected to have higher fidelity for data quality, be more convenient and quality user experience. The wearable solution may be used as an alternative to current symptom diary apps and certainly paper-based symptom diaries.

As described above, consumer-grade, already mass manufactured, reprogrammable sensor tags by MEEBLUE™ (www.meeblue.com) may be used for the wearable solution. Of course, these sensor tags would require custom applications with custom code firmware for the end application. A consumer grade Bluetooth sensor tag that can seamlessly connect to different smartphones running on different operating systems (for example, iOS/Android) can connect robustly with all kinds of smartphones. Currently, there is no similar wearable device on the market that can passively capture stool frequency.

The wearable bowel activity symptom diary solution according to various embodiments of the present disclosure has two advantages over existing technologies. The first is over other long-term wearables such as the ACTIVPAL™ sensor tag which is also an ultra-thin sensor tag worn on the thigh for an extended observation study. The ACTIVPAL™ has two critical flaws for this use case that renders it useless: 1) it only has an accelerometer so cannot distinguish between just sitting and toilet sitting, and 2) it cannot connect wirelessly, and thus, cannot push for Just-in-Time symptom logging on an app. The second is over symptom diary apps where users manually log a bowel passing activity. The burden is still on the patient to remember to use the app consistently and be able to accurately reflect on time and duration of each bowel passing activity, which necessarily will create a less streamlined experience and potential inaccuracies in the log. Our solution addresses these shortcomings of the prior art solutions by introducing a wearable tag that can automatically demarcate bowel passing frequency and duration while enabling just-in-time reflections around food intake, discomforts/pain, and stool consistency and color because the tag can communicate that a bowel passing activity just happened to the phone in a timely fashion. All of this is achieved in the same ultra-thin, waterproof, month-long battery life form factor.

### Bowel Tracker System

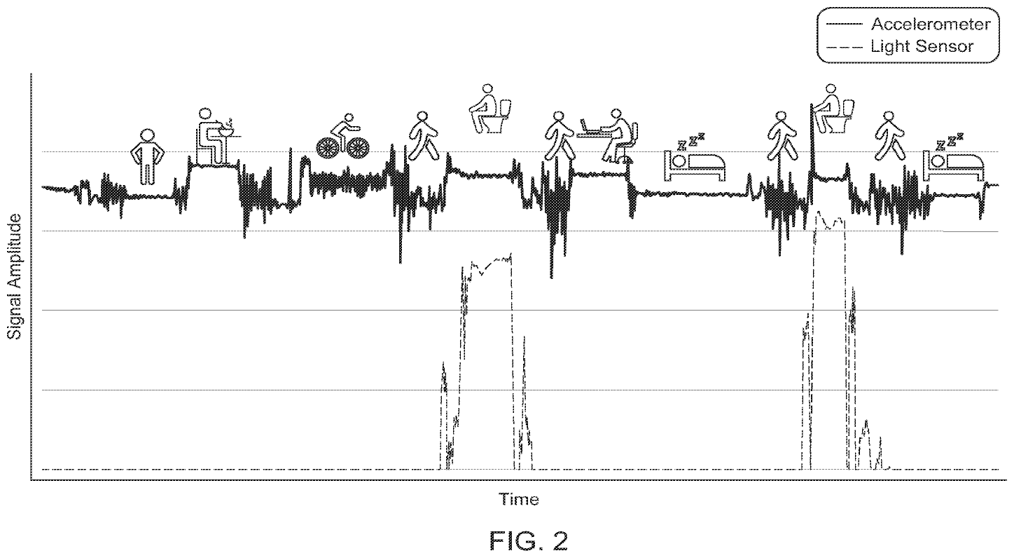

To better help the user record their bowel activities, a bowel tracker system provides features that automate the process from bowel activity detection to information logging. Those features are: (1) bathroom activity detection by the sensor tag 100: the bowel tracker system detecting bathroom activity using the detection algorithm. Referring to FIG. 9, in some embodiments, and the toilet seated position of the person is detected by the accelerometer when the user's thigh wearing the tag 100 and/or the tag 100 is parallel to the ground. In some embodiments, the threshold detected by the accelerometer to be determined as toilet seating is +/− about 10 degrees parallel to the ground. In some embodiments, the detected angle with respect to the ground should be maintained at least for a threshold duration. For example, the threshold duration is 10 seconds. In some embodiments, the threshold duration is 20 seconds, 30 seconds, 40 seconds, 50 seconds, or 1 minute. In some embodiments, the threshold duration is 2 minute, 3 minute, 4 minute, or 5 minute. In some embodiments, in addition to the toilet seating detected by the accelerometer of the tag 100, at least one of the following conditions should be met to determine the toilet seating as a toilet activity. A first condition is that the value of light detected by the light sensor of the tag 100 is greater than a threshold value. A second condition is that the distance between the tag 100 and the toilet tag 100 is less than a threshold distance. For example, the threshold distance is 1 meter. In some embodiments, the threshold distance is 0.9 meter, 0.8 meter, 0.7 meter, 0.6 meter, or 0.5 meter. In some embodiments, the detected distance should be maintained at least for a threshold duration. For example, the threshold duration is 10 seconds. In some embodiments, the threshold duration is 20 seconds, 30 seconds, 40 seconds, 50 seconds, or 1 minute. In some embodiments, the threshold duration is 2 minute, 3 minute, 4 minute, or 5 minute. A third condition is detection of toilet flush by microphone(s) of at least one of the tag 100 and external device 200. In some embodiments, the toilet tag 300 includes a microphone configured to detect the sound of toilet flush. One, two, or all of the three conditions should be satisfied in addition to the toilet seating detected by the accelerometer of the tag 100 to determine the toilet seating as a toilet activity. Once a bathroom activity is detected, a notification will be pushed to the user (FIG. 4).

(2) Referring to FIG. 9, bathroom activity notification is notified via the external device 200 such as smartphone 200a. In some embodiments, the activity notification is notified via a smartwatch 200b. The app will inform the user about the bathroom activity it detects and ask the user if they have done such a bathroom activity via the smartphone 200a or smartwatch 200b. In some embodiments, the user responds to whether toilet activity is bowel movement or not. The user needs to reply to the notification through the action buttons “Yes” or “No” on the notification, as exemplified in FIG. 9. If the user replies “Yes”, the app will take them to a survey and ask them questions relevant to the past bathroom activity (FIG. 4).

(3) Bathroom activity survey: there are several aspects of data being recorded for the bathroom activity. For example, the data includes the time when the bathroom activity finished, the duration of this bathroom activity, the stool form, the presence of blood, the level of urgency, the level of strain, and a note the user wants to leave for this bowel activity. The bathroom activity survey can be performed via the smartphone 200a or smartwatch 200b.

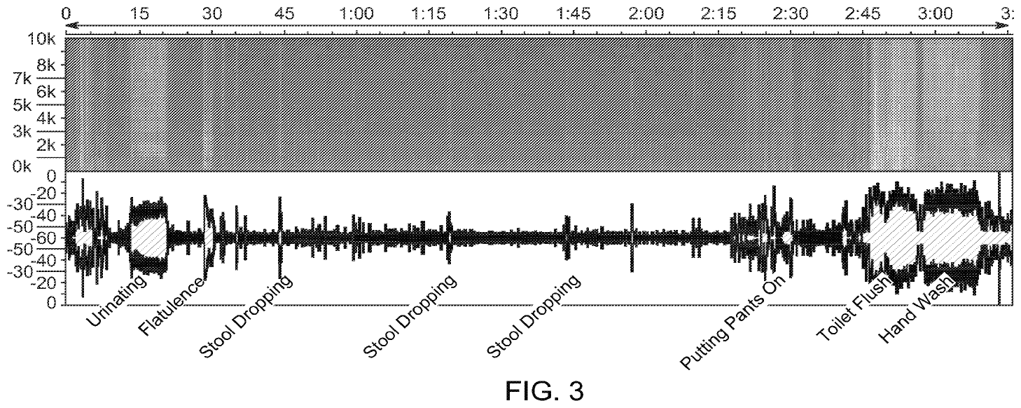

(4) Activity logs: all the user responses will be recorded and saved into a database. The home page of the app will be auto-populated by the activity logs stating if the user has done a bathroom activity. For example, the activity logs is displayed on the smartphone 200a, as shown in FIG. 5.

(5) Background task running: once the sensor tag 100 is connected, even if the user leaves the app in the background, the app will still keep running and detect any bathroom activity that occurs.

### Setting Up the Bowel Tracker System for Daily Bowel Activity Tracking

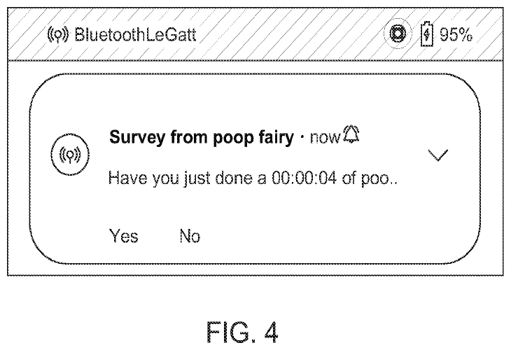

First, open the app on the mobile device 200 and turn on the tag 100. Attach the tag 100 on the leg, as shown in FIG. 6. The sensor tag 100 must be attached to a position on the leg that it can be fully exposed during the bowel activity. For example, the sensor tag 100 is taped to the thigh.

In some embodiments, a toilet tag 300 is provided so that the toilet tag 300 is attached to a toilet used most frequently, for example, at home. The sensor tag 100 is configured to detect or communicate with the toilet tag 300 such that a proximity between them can be determined. When the sensor tag 100 is within a predetermined distance from the toilet tag 300, the possibility of toilet seating is increased significantly. In some embodiments, the toilet tag 300 includes a microphone to detect toilet flush. In some embodiments, the toile tag 300 is further configured to communicate with the smartphone 200a or smartwatch 200b.

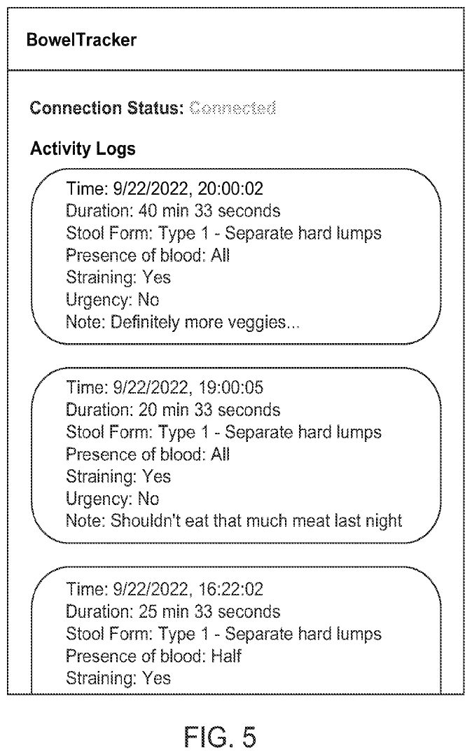

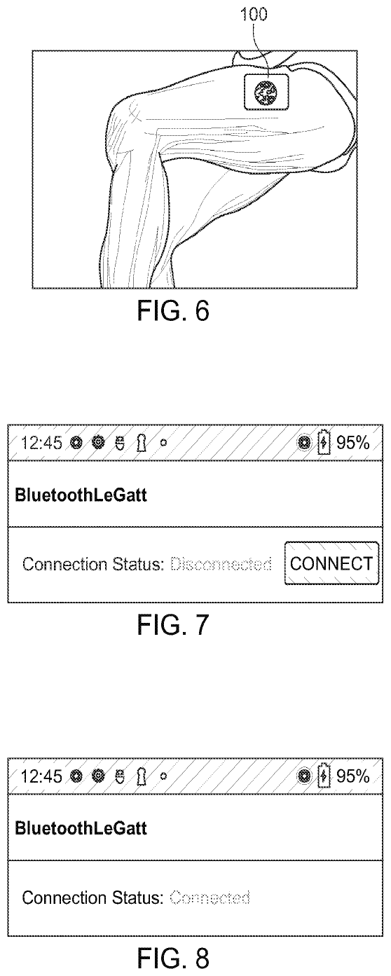

Referring to FIG. 7, to set up the connection with the sensor tag 100, the “Connect” button is clicked on the homepage screen displayed via a display of the mobile device 200. Once the connection is established between the sensor tag 100 and the mobile device 200, the connection status is updated as “Connected,” as exemplified in FIG. 8. In some embodiments, connection is also needs be set up between the sensor tag 100, the mobile device 200, and/or toilet tag 300, if the toilet tag 300 is to be used.

Optionally, calibrate the standing/sitting reading by doing standing/sitting action while having the sensor tag 100 on. Preferably, the standing/sitting action is performed by the subject at the primary toilet that the subject usually uses. This will determine a threshold angle for the accelerometer of the sensor tag 100 to detect to determine a toilet seating position. For example, the threshold is determined as a value close to +/− about 10 degrees parallel to the ground. However, the threshold value may be different depending on the user. After the calibration is done, the app will automatically come back to the homepage. After those settings are done, the user can leave the home page and put the app in the background.

### Use of the Bowel Tracker System to Detect Bowel Activity

The user sits on the toilet and use the bathroom. After finishing, confirm the notification that popped up on the display of the mobile device 200 and finish the survey. The log of this bowel tracker will be recorded and displayed on the home page of the app. The user can now leave the home page and put the app in the background. The log history is presented on the home page, and the screen displayed on the display of the mobile device 200 can be scrolled to check the full history.

### Methodology for the Bowel Tracker System

The BLE sensor tag 100 is attached on the user's upper leg, which is expected to be covered by the user's pants/cloth. During a bathroom activity, the pants/cloth will be taken off and the sensor tag 100 will naturally be exposed to the environmental light. The mobile app will lively receive the data and run the detection algorithm to determine if a bathroom activity happened and would ask the user to evaluate the bathroom activity accordingly. As discussed above, the sensor tag 100 may not be exposed to the environmental light during the nighttime or when the subject does not turn on the light in the bathroom. In this case, a different methodology will be used for the bowel tracker system, as discussed above.

The data communication between the sensor tag 100 and the app installed on the mobile device 200, for example smartphone 200a, is built through the Bluetooth Low-Energy (BLE) protocol. All the relative data (e.g., light, angular acceleration, acceleration) can be received in real-time and provided to the app for data processing.

Based on people's normal bathroom activity behavior, a detection algorithm is designed. The detection algorithm marks any light exposure signal passing a certain threshold as a potential bathroom activity's starting point. Then the detection algorithm would begin to evaluate the current bathroom activity until an endpoint of the activity occurs or when the evaluation determines it is not a bathroom activity. The endpoint happens when the light signal goes below a certain light threshold for a certain duration of time and the user has transited to a standing gesture from sitting.

During the evaluation of the current bathroom activity, we use live acceleration, angular acceleration, sitting/standing gestures, and light to track if there is actually a bathroom activity going on. There are at least three criteria used in the detection algorithm: (1) duration of the bathroom activity: if the light exposure lasts <10 seconds, we do not consider it a bathroom activity although the threshold duration of the light exposure may be greater in some embodiments, for example, about 15 seconds, 20 second, 25 seconds, 30 seconds, or up to 1 minute; (2) sitting/standing gesture of the user: we use the calibrated data of the standing/sitting gesture as a baseline to infer the user's gesture. The user is expected to remain sitting on a toilet during the bathroom activity. In some embodiments, the user is expected to remain sitting for at least 10 seconds for the seating to be considered as a toilet activity. In some embodiments, the duration of the seating should be greater than about 20 second, 30 seconds, 40 seconds, 50 seconds, or 1 minute for the seating to be considered as a toilet activity. This can be deployed to prevent false positive recognition of bathroom action. (3) proximity to a toilet BLE tag 300 placed on the toilet. If the user is seated and within 1 meter of a BLE toile tag 300, this will be used to determine use of the toilet. See FIG. 9. The proximity to the toilet tag 300 may be a threshold distance range of at least 0.5 meter to 1 meter between the sensor tag 100 and the toilet tag 300, between the sensor tag 100 and the smartwatch 200b, or both. In some embodiments, the threshold distance is 1 meter, as exemplified in C of FIG. 9.

Overall, using the bowel tracker system, a successful bathroom activity needs to satisfy all the requirements including (1) the sensor tag 100 exposes to light (the starting point) although there are exceptions of toilet seating in the darkness, as discussed above. Referring to B of FIG. 9, the light value measured by the sensor tag 100 is one of a plurality of conditions for detecting toilet activity, and toilet activity may be detected even in the absence of the light exposure in some circumstances; (2) the light exposure lasts for a long enough duration if the light exposure is used as a condition for detecting toilet activity; (3) the user does not move dramatically (compared to walking or jumping) when performing the bathroom activity, as detected by the accelerometer of the sensor tag 100 or smartwatch 200b; (4) the sensor tag 100 does not expose to the light for a long enough duration (the ending point).

According to various embodiments of the present disclosure, a wearable device to be worn by a user includes an accelerometer configured to detect changes in positions of the user wearing the wearable device; a light sensor configured to detect changes in intensities of light; and a transmitter configured to communicate with an external device. According to various aspects of the present disclosure, the wearable device is configured to detect when the user is sitting with the user's lower body undressed based on the changes detected by the accelerometer and light sensor; capture stool frequency data based on the detected sitting by the user; and transmit the captured stool frequency data to the external device.

According to some embodiments of the present disclosure, the captured stool frequency data is transmitted to the external device to be processed by the external device and/or to be displayed at the external device. According to some embodiments of the present disclosure, the stool frequency data captured and transmitted by the wearable device and processed by the external device is presented to the user by being displayed at the external device. According to some embodiments of the present disclosure, capturing, transmitting, processing and displaying the stool frequency data are performed passively or automatically without requiring the user's direct or manual input via the wearable device and/or external device.

According to some embodiments of the present disclosure, the captured stool frequency data is transmitted from the wearable device to the external device when the wearable device and the external device are within a threshold distance allowing wireless communication between the wearable device and the external device. According to some embodiments of the present disclosure, when the wearable device and the external device are not within the threshold distance at the time of capturing the stool frequency data, the wearable device is further configured to transmit the captured stool frequency data to the external device next time when the wearable device and the external device are within the threshold distance. According to some embodiments of the present disclosure, the wearable device further includes a memory configured to store the captured stool frequency data. According to some embodiments of the present disclosure, the captured stool frequency data is stored at the memory until the captured stool frequency data is transmitted to the external device.

According to some embodiments of the present disclosure, the wearable device further includes a processor configured to receive data including the detected changes from the accelerometer and light sensor; and determine whether the user is sitting on a toilet based on the received data. According to some embodiments of the present disclosure, the processor is further configured to capture stool frequency, timing, and duration of the user.

According to some embodiments of the present disclosure, the wearable device includes a tag worn on a thigh of the user. According to some embodiments of the present disclosure, the tag is worn at an upper portion of the thigh. According to some embodiments of the present disclosure, the tag is less than 0.5 cm thick.

According to some embodiments of the present disclosure, the transmitter includes a Bluetooth transmitter configured to communicate with the external device paired with the Bluetooth transmitter and/or positioned within a specified distance from the wearable device.

According to some embodiments of the present disclosure, the user is a patient with a bowel disorder or gastrointestinal disease. According to some embodiments of the present disclosure, the bowel disorder includes Crohn's disease and, ulcerative colitis, and functional bowel disorders (FBD) including irritable bowel syndrome and functional dyspepsia.

According to some embodiments of the present disclosure, the wearable device further includes a microphone, and the processor is further configured to receive acoustic data sensed by the microphone and/or acoustic data sensed by a microphone of the external device; and determine whether the user is sitting on the toilet based on the received acoustic data in addition to the changes detected by the accelerometer and light sensor.

According to some embodiments of the present disclosure, the acoustic data are robustly identified using a neural network model tuned for acoustic classification running on the external device; and a neural network model utilizes transfer learning to first pre-train a convolutional neural network (CNN) with a large corpus of labeled acoustics data.

According to some embodiments of the present disclosure, the wearable device of further includes an audio output unit or a speaker configured to output a notification when the processor determines that the user has been in a seating position for more than a preset period of time.

According to some embodiments of the present disclosure, the wearable device further includes an input unit or a button configured to receive an input from the user in response to the notification output from the audio output unit or speaker, the input confirming that the user is actually having a bowel movement.

According to some embodiments of the present disclosure, the processor is further configured to train algorithm for demarcating bowel movement events in response to the input, using machine learning and statistical modeling, verification performed by the external device. According to some embodiments of the present disclosure, the processor is further configured to cause the transmitter to transmit the captured stool frequency, timing, and duration of the user to the external device comprising a smartwatch or smartphone.

According to some embodiments of the present disclosure, the external device is configured to execute an application to process the captured stool frequency data received from the wearable device. According to some embodiments of the present disclosure, the external device includes a display and is further configured to initiate a push notification to the user, noting a bowel movement was detected; and present an ecological momentary assessment (EMA) on the display.

According to some embodiments of the present disclosure, the external device is further configured to display visual information on the display in response to the processed stool frequency data. According to some embodiments of the present disclosure, the visual information includes a visual picker for Bristol Stool Scale and select questionnaire around food/fluid intake and discomfort/pain.

According to various embodiments of the present disclosure, a method for monitoring bowel habits of a subject by a wearable device worn by the subject includes detecting, by an accelerometer of the wearable device, changes in positions of the subject; detecting, by a light sensor of the wearable device, changes in intensities of light; detecting, by a processor of the wearable device, when the subject is sitting with the subject's lower body undressed based on the changes detected by the accelerometer and light sensor; capturing, by the processor, stool frequency data based on the detected sitting by the subject; and transmitting, via a transmitter of the wearable device, the captured stool frequency data to the external device.

According to some embodiments of the present disclosure, the method further includes receiving, by the processor, data including the detected changes from the accelerometer and light sensor; and determining, by the processor, whether the subject is sitting on a toilet based on the received data. According to some embodiments of the present disclosure, the method further includes capturing stool frequency, timing, and duration of the subject.

According to some embodiments of the present disclosure, the wearable device includes a tag worn on a thigh of the subject. According to some embodiments of the present disclosure, the tag is worn at an upper portion of the thigh. According to some embodiments of the present disclosure, the tag is less than 0.5 cm thick.

According to some embodiments of the present disclosure, the transmitter includes a Bluetooth transmitter configured to communicate with the external device paired with the Bluetooth transmitter and/or positioned within a specified distance from the wearable device.

According to some embodiments of the present disclosure, the subject is a patient with a bowel disorder or gastrointestinal disease. According to some embodiments of the present disclosure, the bowel disorder includes Crohn's disease, ulcerative colitis, and functional bowel disorders (FBD) comprising irritable bowel syndrome and functional dyspepsia.

According to some embodiments of the present disclosure, the wearable device further includes a microphone, and the method further includes receiving acoustic data sensed by the microphone and/or acoustic data sensed by a microphone of the external device; and determining whether the subject is sitting on the toilet based on the received acoustic data.

According to some embodiments of the present disclosure, the acoustic data are robustly identified using a neural network model tuned for acoustic classification running on the external device; and a neural network model utilizes transfer learning to first pre-train a convolutional neural network (CNN) with a large corpus of labeled acoustics data.

According to some embodiments of the present disclosure, the wearable device further includes an audio output unit or a speaker, and the method further includes outputting, via the audio output unit or speaker, a notification when the processor determines that the subject has been in a seating position for more than a preset period of time.

According to some embodiments of the present disclosure, the wearable device further includes an input unit or a button, and the method further includes receiving an input from the subject, via the input unit or button, in response to the notification output from the audio output unit or speaker, the input confirming that the subject is actually having a bowel movement.

According to some embodiments of the present disclosure, the method further includes training algorithm for demarcating bowel movement events in response to the input, using machine learning and statistical modeling, verification performed by the external device.

According to some embodiments of the present disclosure, the method further includes transmitting, via the transmitter, the captured stool frequency, timing, and duration of the subject to the external device comprising a smartwatch or smartphone.

According to some embodiments of the present disclosure, the external device is configured to execute an application to process the captured stool frequency data received from the wearable device. According to some embodiments of the present disclosure, the external device includes a display and is further configured to initiate a push notification to the subject, noting a bowel movement was detected; and present an ecological momentary assessment (EMA) on the display.

According to some embodiments of the present disclosure, the external device is further configured to display visual information on the display in response to the processed stool frequency data. According to some embodiments of the present disclosure, the visual information includes a visual picker for Bristol Stool Scale and select questionnaire around food/fluid intake and discomfort/pain.

According to some embodiments of the present disclosure, the captured stool frequency data is transmitted from the wearable device to the external device when the wearable device and the external device are within a threshold distance allowing wireless communication between the wearable device and the external device. According to some embodiments of the present disclosure, when the wearable device and the external device are not within the threshold distance at the time of capturing the stool frequency data, the wearable device is further configured to transmit the captured stool frequency data to the external device next time when the wearable device and the external device are within the threshold distance.

According to various embodiments of the present disclosure, a system for monitoring bowel habits of a subject includes a wearable device to be worn by the subject and a smart device configured to communicate with the wearable device wirelessly. In one aspect of the present disclosure, the wearable device includes an accelerometer configured to detect changes in positions of the subject wearing the wearable device; a light sensor configured to detect changes in intensities of light; and a transmitter configured to communicate with the smart device. According to some embodiments of the present disclosure, the wearable device is configured to detect when the subject is sitting with the subject's lower body undressed based on the changes detected by the accelerometer and light sensor; capture stool frequency data based on the detected sitting by the subject; and transmit the captured stool frequency data to the smart device. According to some embodiments of the present disclosure, the smart device is further configured to execute an app to process the stool frequency data received from the wearable device.

According to some embodiments of the present disclosure, the wearable device includes a tag worn on a thigh of the subject; and the smart device includes a smartwatch or smartphone paired with the transmitter and/or positioned within a specified distance from the wearable device.

According to some embodiments of the present disclosure, the subject is a patient with a bowel disorder or gastrointestinal disease. According to some embodiments of the present disclosure, the bowel disorder comprises Crohn's disease and ulcerative colitis.

According to some embodiments of the present disclosure, the wearable device further includes a microphone, and the wearable device is further configured to receive acoustic data sensed by the microphone and/or acoustic data sensed by a microphone of the smart device; and determine whether the subject is sitting on the toilet based on the received acoustic data in addition to the changes detected by the accelerometer and light sensor.

According to some embodiments of the present disclosure, the acoustic data are robustly identified using a neural network model tuned for acoustic classification running on the smart device; and a neural network model utilizes transfer learning to first pre-train a convolutional neural network (CNN) with a large corpus of labeled acoustics data.

According to some embodiments of the present disclosure, the wearable device further includes an audio output unit or a speaker configured to output a notification when the subject has been determined to be in a seating position for more than a preset period of time.

According to some embodiments of the present disclosure, the wearable device further includes an input unit or a button configured to receive an input from the subject in response to the notification output from the audio output unit or speaker, the input confirming that the subject is actually having a bowel movement.

According to some embodiments of the present disclosure, at least one of the wearable device or the smart device is further configured to train algorithm for demarcating bowel movement events in response to the input, using machine learning and statistical modeling, verification performed by the smart device.

According to some embodiments of the present disclosure, the transmitter is further configured to transmit the captured stool frequency, timing, and duration of the subject to the smart device.

According to some embodiments of the present disclosure, the smart device is configured to execute an application to process the captured stool frequency data received from the wearable device. According to some embodiments of the present disclosure, the smart device includes a display and is further configured to initiate a push notification to the subject, noting a bowel movement was detected; and present an ecological momentary assessment (EMA) on the display.

According to some embodiments of the present disclosure, the smart device is further configured to display visual information on the display in response to the processed stool frequency data. According to some embodiments of the present disclosure, the visual information includes a visual picker for Bristol Stool Scale and select questionnaire around food/fluid intake and discomfort/pain.

According to some embodiments of the present disclosure, the captured stool frequency data is transmitted to the smart device to be processed by the smart device and/or to be displayed at the smart device. According to some embodiments of the present disclosure, the stool frequency data captured and transmitted by the wearable device and processed by the smart device is presented to the subject by being displayed at the smart device. According to some embodiments of the present disclosure, capturing, transmitting, processing and displaying the stool frequency data are performed passively or automatically without requiring the subject's direct or manual input via the wearable device and/or smart device.

According to some embodiments of the present disclosure, the captured stool frequency data is transmitted from the wearable device to the smart device when the wearable device and the smart device are within a threshold distance allowing wireless communication between the wearable device and the smart device. According to some embodiments of the present disclosure, when the wearable device and the smart device are not within the threshold distance at the time of capturing the stool frequency data, the wearable device is further configured to transmit the captured stool frequency data to the smart device next time when the wearable device and the smart device are within the threshold distance.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

1. A bowel tracker system for monitoring bowel habits of a subject, the system comprising:
a wearable device to be worn by the subject; and
a smart device configured to communicate with the wearable device wirelessly,
wherein the wearable device comprises:
an accelerometer configured to detect changes in positions of the subject wearing the wearable device;
a light sensor configured to detect changes in intensities of light; and
a transmitter configured to communicate with the smart device,
wherein the wearable device is configured to:
detect when the subject is sitting with the subject's lower body undressed based on the changes detected by at least one of the accelerometer and the light sensor;
capture stool frequency data based on the detected sitting by the subject; and
transmit the captured stool frequency data to the smart device, and
wherein the smart device is further configured to execute an application to process the stool frequency data received from the wearable device.

2. The system of claim 1, wherein:
the wearable device comprises a tag worn on a thigh of the subject, wherein the bowel tracker system is configured to detect that the subject is seating on a toilet when the tag is substantially parallel to the ground for at least a threshold duration; and/or
the smart device comprises a smartwatch or smartphone paired with the transmitter and/or positioned within a specified distance from the wearable device, wherein at least one of the smartwatch and the smartphone is configured to detect toilet flush via a microphone integrated therein, the detected toilet flush being used for determination of a bowel movement activity by the subject.

3. (canceled)

4. The system of claim 2, further comprising a toilet tag attachable to a toilet, wherein the toilet tag is configured to communicate with at least one of the wearable device and the smart device, and wherein a distance between the toilet tag attached to the toilet and the wearable device and/or the smart device is used to determine toilet seating of the subject.

5. The system of claim 4, wherein a threshold distance between the toilet tag attached to the toilet and the wearable device is 1 meter such that the subject is determined to be in a toilet seating position when the distance is less than 1 meter.

6. (canceled)

7. The system of claim 2, wherein the wearable device further comprises a microphone, an audio output unit or a speaker, an input unit or a button, and/or a memory,
wherein the smart device comprises a display,
wherein the wearable device is further configured to:
receive acoustic data sensed by the microphone and/or acoustic data sensed by a microphone of the smart device; and
determine whether the subject is sitting on the toilet based on the received acoustic data in addition to the changes detected by the accelerometer and light sensor,
wherein the audio output unit or speaker is configured to output a notification when the subject has been determined to be in a seating position for more than a preset period of time,
wherein the input unit or button is configured to receive an input from the subject in response to the notification output from the audio output unit or speaker, the input confirming that the subject is actually having a bowel movement,
wherein the memory is configured to store the captured stool frequency data, and/or the captured stool frequency data is stored at the memory until the captured stool frequency data is transmitted to the smart device, and/or
wherein the smart device is further configured to initiate a push notification to the subject, noting a bowel movement was detected; and present an ecological momentary assessment (EMA) on the display.

8. The system of claim 7, wherein:
the acoustic data are robustly identified using a neural network model tuned for acoustic classification running on the smart device; and
a neural network model utilizes transfer learning to first pre-train a convolutional neural network (CNN) with a large corpus of labeled acoustics data.

9-10. (Canceled)

11. The system of claim 7, wherein at least one of the wearable device or the smart device is further configured to train algorithm for demarcating bowel movement events in response to the input, using machine learning and statistical modeling, verification performed by the smart device.

12. The system of claim 1, wherein;
the transmitter is further configured to transmit the captured stool frequency, timing, and duration of the subject to the smart device;
the captured stool frequency data is transmitted to the smart device to be processed by the smart device and/or to be displayed at the smart device;
the stool frequency data captured and transmitted by the wearable device and processed by the smart device is presented to the subject by being displayed at the smart device; and/or
the smart device is further configured to display visual information on the display in response to the processed stool frequency data, the visual information.

13-14. (canceled)

15. The system of claim 12, wherein the visual information comprises a visual picker for Bristol Stool Scale and select questionnaire around food/fluid intake and discomfort/pain.

16-17. (canceled)

18. The system of claim 12, wherein the capturing, transmitting, processing, and displaying the stool frequency data are performed passively or automatically without requiring the subject's direct or manual input via the wearable device and/or smart device.

19. The system of claim 12, wherein;
the captured stool frequency data is transmitted from the wearable device to the smart device when the wearable device and the smart device are within a threshold distance allowing wireless communication between the wearable device and the smart device; and/or
when the wearable device and the smart device are not within the threshold distance at the time of capturing the stool frequency data, the wearable device is further configured to transmit the captured stool frequency data to the smart device next time when the wearable device and the smart device are within the threshold distance.

20-22. (canceled)

23. A method for monitoring bowel habits of a subject by a bowel tracker system including a wearable device worn by the subject, the method comprising:
detecting, by an accelerometer of the wearable device, changes in positions of the subject;
detecting, by a light sensor of the wearable device, changes in intensities of light;
detecting, by a processor of the wearable device, when the subject is sitting with the subject's lower body undressed based on the changes detected by the accelerometer and light sensor;
capturing, by the processor, stool frequency data based on the detected sitting by the subject; and
transmitting, via a transmitter of the wearable device, the captured stool frequency data to an external device.

24. The method of claim 23, further comprising:
receiving, by the processor, data including the detected changes from the accelerometer and light sensor;
determining, by the processor, whether the subject is sitting on a toilet based on the received data;
capturing stool frequency, timing, and duration of the subject; and/or
determining, by the processor, a toilet seated position in response to:
the accelerometer detecting that the wearable device or tag worn by the subject is substantially parallel to the ground; and/or
a smartwatch worn by the subject detecting that the subject is seated or the smartwatch being detected to be substantially parallel to the ground.

25. (canceled)

26. The method of claim 23, wherein:
the wearable device comprises a tag worn on a thigh or at an upper portion of the thigh of the subject; and/or
the transmitter comprises a BLUETOOTH transmitter configured to communicate with the external device paired with the BLUETOOTH transmitter and/or positioned within a specified distance from the wearable device.

27-29. (canceled)

30. The method of claim 23, wherein:
the subject is a patient with a bowel disorder or gastrointestinal disease, and wherein the bowel disorder comprises Crohn's disease, ulcerative colitis, and functional bowel disorders (FBD) comprising irritable bowel syndrome and chronic constipation; and/or
the subject is determined to be in a sitting position with the subject's lower body undressed when the light intensity detected by the light sensor of the wearable device is greater than a light threshold value and when the accelerometer detects that the wearable device or tag worn by the subject is substantially parallel to the ground.

31. (canceled)

32. The method of claim 23, wherein the wearable device further comprises a microphone, the method further comprising:
receiving acoustic data sensed by the microphone and/or acoustic data sensed by a microphone of the external device; and
determining whether the subject is sitting on the toilet based on the received acoustic data in addition to the changes detected by the accelerometer and light sensor.

33. The method of claim 32, wherein:
the acoustic data are robustly identified using a neural network model tuned for acoustic classification running on the external device; and
a neural network model utilizes transfer learning to first pre-train a convolutional neural network (CNN) with a large corpus of labeled acoustics data.

34-35. (canceled)

36. The method of claim 23, wherein the wearable device further comprises an audio output unit or a speaker and/or an input unit or a button, the method further comprising:
outputting, via the audio output unit or speaker, a notification when the processor determines that the subject has been in a seating position for more than a preset period of time;
receiving an input from the subject, via the input unit or button, in response to the notification output from the audio output unit or speaker, the input confirming that the subject is actually having a bowel movement;
training algorithm for demarcating bowel movement events in response to the input, using machine learning and statistical modeling, verification performed by the external device; and/or transmitting, via the transmitter, the captured stool frequency, timing, and duration of the subject to the external device comprising a smartwatch or smartphone.

37. (canceled)

38. The method of claim 23, wherein:
the external device is configured to execute an application to process the captured stool frequency data received from the wearable device; and/or
the external device comprises a display and is further configured to:
initiate a push notification to the subject, noting a bowel movement was detected; and
present an ecological momentary assessment (EMA) on the display.

39. (canceled)

40. A wearable device configured to be worn by a user, the wearable device comprising:
an accelerometer configured to detect changes in positions of the user wearing the wearable device;
a light sensor configured to detect changes in intensities of light;
a transmitter configured to communicate with an external device; and
a processor, configured to:
receive data including the detected changes from the accelerometer and light sensor;
determine whether the user is sitting on a toilet based on the received data;
detect when the user is sitting with the user's lower body undressed based on the changes detected by the accelerometer and light sensor;
capture stool frequency data based on the detected sitting by the user; and
cause the transmitter to transmit the captured stool frequency data to the external device.

41-42. (canceled)

43. The wearable device of claim 40, wherein:
the wearable device comprises a tag worn on a thigh of the user;
the tag is worn at an upper portion of the thigh; and/or
the tag is less than 0.5 cm thick.

44-48. (canceled)

49. The wearable device of claim 40, further comprising:
a microphone, wherein the processor is further configured to:
receive acoustic data sensed by the microphone and/or acoustic data sensed by a microphone of the external device; and
determine whether the user is sitting on the toilet based on the received acoustic data in addition to the changes detected by the accelerometer and light sensor;
an audio output unit or a speaker configured to output a notification when the processor determines that the user has been in a seating position for more than a preset period of time; and/or
an input unit or a button configured to receive an input from the user in response to the notification output from the audio output unit or speaker, the input confirming that the user is actually having a bowel movement.

50. The wearable device of claim 49, wherein:
responsive to the acoustic data being indicative of the user having a bowel movement, determining that the user is having a bowel movement regardless of whether the changes detected by at least the light sensor indicate that the user is sitting on the toilet;
the acoustic data are robustly identified using a neural network model tuned for acoustic classification running on the external device; and/or
a neural network model utilizes transfer learning to first pre-train a convolutional neural network (CNN) with a large corpus of labeled acoustics data.

51-57. (canceled)

58. The wearable device of claim 40, wherein;
the changes detected by light sensor includes a change in a light intensity detected by the light sensors exceeds a first threshold, and the changes detected by the accelerometer includes a decrease in a height position of the user detected by the accelerometer exceeds a second threshold;
the change in the light intensity exceeds the first threshold by a period of at least two seconds or at least three seconds during which period the decrease in the height position also exceeds the second threshold, and/or
the first threshold is at least 20% or at least 30% or at least 40% or at least 50% or at least 60% or least 70% or at least 80% or at least 90% relative to a light intensity before the detected change.

59-60. (canceled)

61. The wearable device of claim 40, wherein a toilet seated position is detected by the accelerometer when the wearable device or tag worn by the user is substantially parallel to the ground.
